(19) United States
(12) Patent Application Publication
(10) Pub. No.: US 2011/0034889 A1
(43) Pub. Date: Feb. 10, 2011

(54) METHOD AND APPARATUS FOR SUPPORTING A DRAINAGE BAG

(76) Inventor: Beverly A. Smith, Largo, FL (US)

Correspondence Address: LARSON AND LARSON, 11199 69TH STREET NORTH, LARGO, FL, 33773, US

(21) Appl. No.: 12/537,545

(22) Filed: Aug. 7, 2009

(51) Int. Cl. A61F 5/44

(52) U.S. Cl. 604/327

(57) ABSTRACT

An application for a drainage bag support includes a belt that has at least one way to engaging with an animate object (person). The belt has an inwardly facing surface that rests against the animate object and an outwardly facing surface facing away from the animate object. At least one fastening station is/are positioned on the outwardly facing surface of the belt. One or more fasteners interface one or more drainage bags to one or more of the fastening stations, thereby supporting the drainage bags by the belt.

### FIELD OF THE INVENTION

This invention relates to the field of medicine and more particularly to an apparatus for supporting a drainage device.

### BACKGROUND

After a surgery, the individual often has one or more drainage bags for draining the incision. The drainage bags are connected to tubes that interface with the incision, thereby draining any seepage from the incision through the tube(s) and into the drainage bag(s). Many times, the drainage bags are simply supported by the tubes, placing stress on the incision, causing discomfort to the patient and increasing the risk of the tubes being pulled out of the incision.

To improve this situation, the drainage bag(s) are sometimes affixed to the patient's surgical dressing or gown with a safety pin. Attaching to the dressing initially works when the dressing is substantial, but as the patient heals, the size and thickness of the dressing reduces, making it difficult to support the drainage bag(s). Attaching to the gown helps, but there is still the risk of pulling the tubes out of the incision.

Other solutions have been attempted. Healing Enhancements (see www.healingenhancements.com/surgical drainbags.aspx) markets a waist purse that holds one or more drainage bags. This device is similar to a fanny pack, is large and more complicated than is needed.

What is needed is an apparatus that will hold one or more drain bags without undue material.

### SUMMARY OF THE INVENTION

In one embodiment, a drainage bag support is disclosed including a belt that has at least one way to engaging with an animate object. The belt has an inwardly facing surface that rests against the animate object and an outwardly facing surface facing away from the animate object. At least one fastening station is/are positioned on the outwardly facing surface of the belt. One or more fasteners interface one or more drainage bags to one or more of the fastening stations, thereby supporting the drainage bags by the belt. The animate object is preferably a person and the belt is typically installed around the waist of the person, though it is anticipated that the present invention is equally applicable to other animate objects (e.g. livestock, pets, wild animals, etc.) and/or other areas of the animate objects (e.g. around a neck, arm, leg, wing, tail, fin, etc.).

In another embodiment, a method of supporting a drainage bag is disclosed including providing a drainage bag support that has a belt and one or more fastening stations. The belt is made of a continuous loop of a stretchable material and has an inwardly facing surface facing toward the center of the continuous loop and an outwardly facing surface facing away from the center of the continuous loop. The fastening station(s) are positioned on the outwardly facing surface of the belt. The method includes stretching the belt to engage the belt with an area of an animate object then releasing the belt, the belt therefore contracting around the area of the animate object do to the force of contraction of the belt. Once released and in place, one or more drainage bags are affixed to one or more of the fastening stations.

In another embodiment, a method of supporting a drainage bag is disclosed including providing a drainage bag support that includes a belt and at least one fastening station. The belt has an inwardly facing surface and an outwardly facing surface facing opposite of the inwardly facing surface. The belt also has hook and loop material on the outwardly facing surface at a first end of the belt and mating hook and loop material on the inwardly facing surface at a second end of the belt. The fastening stations are positioned on the outwardly facing surface of the belt. The method includes passing the belt around an area of an animate object (e.g. the waist of a person) then engaging the hook and loop material with the mating hook and loop material, thereby securing the belt around the area of the animate object. Once secured around the area of the animate object, one or more drainage bags are affixed to one or more of the fastening stations.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a perspective view of a system of the prior art.

FIG. 2 illustrates a perspective view of a first embodiment of the present invention.

FIG. 3 illustrates a second perspective view of a first embodiment of the present invention.

FIG. 4 illustrates a perspective view of a second embodiment of the present invention.

FIG. 5 illustrates a perspective view of both embodiments of the present invention using a clip to fasten the drainage bag.

FIG. 6 illustrates a perspective view of both embodiments of the present invention using a hook and loop system to fasten the drainage bag.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

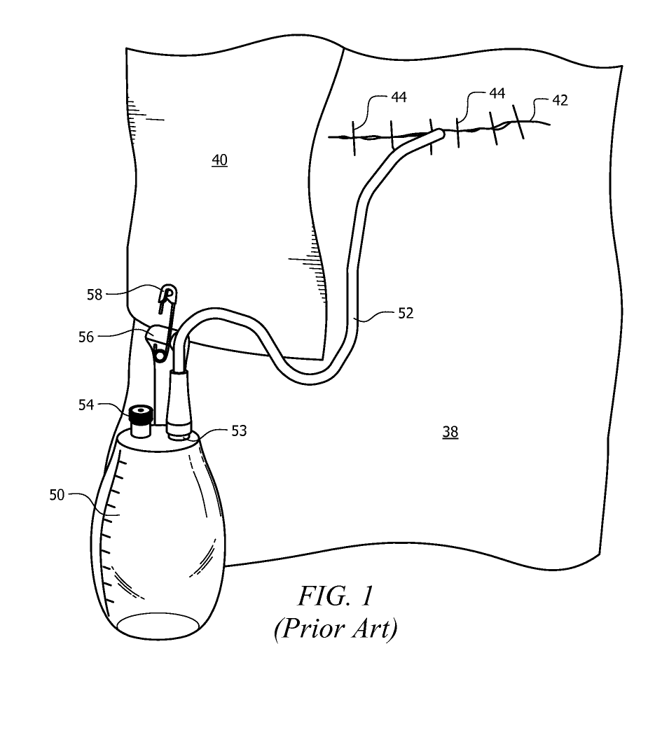

Referring to FIG. 1, a perspective view of a system of the prior art is shown. After an operation, often a drainage bag 50 is interfaced to the wound 42 (shown with sutures 44) through a drainage tube 52 that interfaces with a nipple 53 on the drainage bag. Although shown as a typical drainage bag 50, there are many known shapes and types of drainage bags 50 and the present invention is not limited to any particular type, shape, size or compositions of drainage bag 50. This exemplary drainage bag 50 has a drain 54 used, for example, to empty the contents of the drainage bag. The drainage bag 50 also has an appendage 56 that provides a way to fasten the drainage bag 50 to something. In this example of the prior art, the drainage bag 50 is attached by a safety pin 58 that interfaces with both the appendage 56 and the dressing 40 that covers the wound 42. Note the dressing 40 is not shown covering the wound 42 to illustrate the operation of the drainage bag 50 and drainage tube 52.

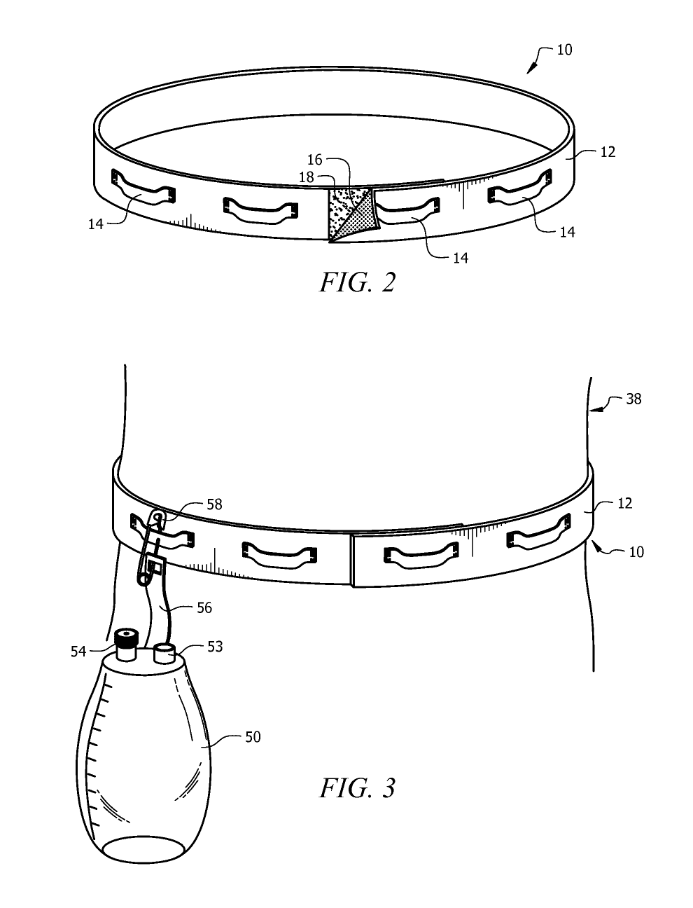

Referring to FIG. 2, a perspective view of a first embodiment of the present invention is shown. The drainage bag support system 10 of the present invention includes a belt 12 having one or more fastening stations 14 on an external surface of the belt 12. The belt 12 is placed around a person's waist, body or appendage and fastened to itself with hook and loop material 16/18, keeping the belt in place around the waist, body or appendage.

Referring to FIG. 3, a second perspective view of a first embodiment of the present invention is shown. The drainage bag support system 10 as described with FIG. 2 is shown positioned around a person's waist 38 and fastened to itself with the hook and loop material 16/18, keeping the belt in place around the waist 38. The drainage bag 50 is pinned to one of the fastening stations 14 with a safety pin 58 or other suitable fastener that interfaces the appendage 56 of the drainage bag 50 to one of the fastening station 14. It is anticipated that multiple drainage bags 50 are fastened to one or more of the fastening stations 14, as needed.

For brevity reasons, the drainage bag drain 54 and nipple 53 are shown. In typical use, a drainage tube 52 (see FIG. 1) is connected at one end to the nipple 53 and at a distal end the drainage tube 52 interfaces with the wound 42 (see FIG. 1) requiring drainage.

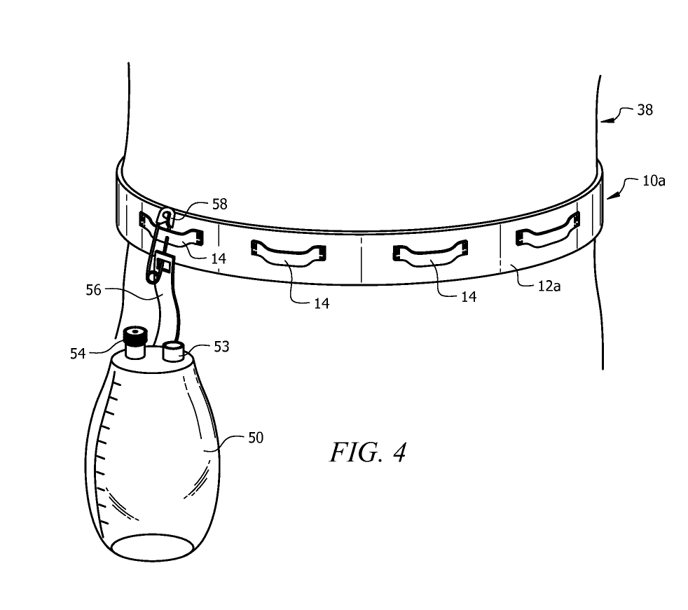

Referring to FIG. 4, a perspective view of a second embodiment of the present invention is shown. The drainage bag support system 10a is shown positioned around a person's waist 38. In this version of the drainage bag support system 10a, the belt portion 12a is stretchable as would be the waist band of underwear. The belt portion 12a is stretched to allow the belt portion 12a to be placed around the person's waist 38 (as shown), body or appendage. Once in place, the elastic action of the belt portion 12a keeps the belt in place around, for example, the person's the waist 38. As in the prior example, the drainage bag 50 is pinned to one of the fastening stations 14 with a safety pin 58 or other suitable fastener that interfaces the appendage 56 of the drainage bag 50 to one of the fastening station 14. It is anticipated that multiple drainage bags 50 are fastened to one or more of the fastening stations 14, as needed.

As in FIG. 3, the drainage bag drain 54 and nipple 53 are shown. In typical use, a drainage tube 52 (see FIG. 1) is connected at one end to the nipple 53 and at a distal end the drainage tube 52 interfaces with the wound 42 (see FIG. 1) requiring drainage.

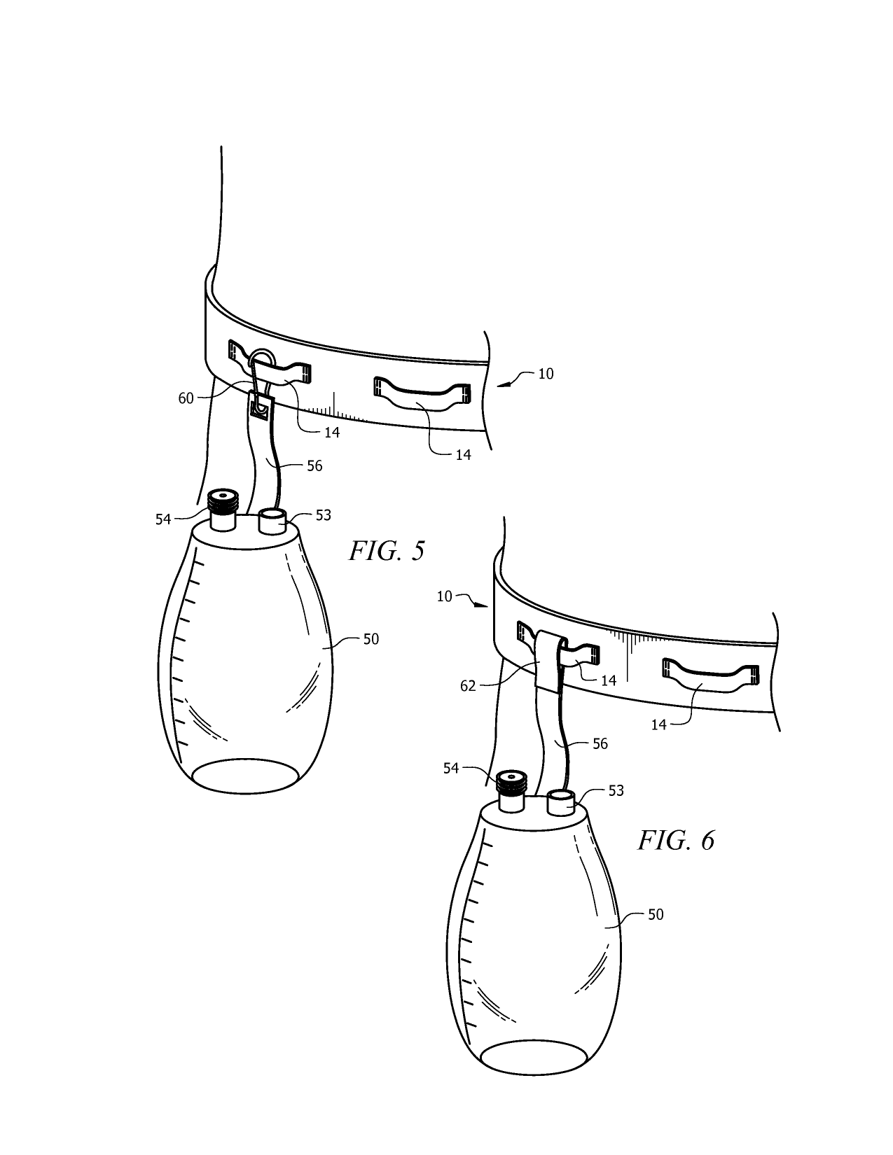

Referring to FIG. 5, a perspective view of both embodiments of the present invention using a clip to fasten the drainage bag is shown. The drainage bag support system 10/10a as described with FIG. 2 is shown positioned around a person's waist 38 and held in place by, for example, the hook and loop material 16/18 or the elastic belt portion 12a, keeping the belt in place around the waist 38. In this example, the drainage bag 50 is clipped to one of the fastening stations 14 with a clip 60, interfacing the appendage 56 of the drainage bag 50 to one of the fastening station 14. It is anticipated that multiple drainage bags 50 are fastened to one or more of the fastening stations 14, as needed, using one or more clips 60.

Referring to FIG. 6, a perspective view of both embodiments of the present invention using a hook and loop system to fasten the drainage bag is shown. The drainage bag support system 10 as described with FIG. 2 is shown positioned around a person's waist 38 and held in place by, for example, the hook and loop material 16/18 or the elastic belt portion 12a, keeping the belt in place around the waist 38. In this example, the drainage bag 50 is secured to one of the fastening stations 14 with a loop 62 on the appendage 56 that fastens to itself as known in the industry. For example, the end portion has hook and loop material that holds to mating hook and loop material on a mid-portion of the appendage 56. Another example uses a snap at the end portion that snaps to a mating snap on a mid-portion of the appendage 56. Another example uses a button on the mid-portion of the appendage 56 that interfaces with a button hole located on the end of the loop. The loop 62 affixes the appendage 56 of the drainage bag 50 to one of the fastening station 14. It is anticipated that multiple drainage bags 50 are fastened to one or more of the fastening stations 14, as needed, using one or more clips 60.

Equivalent elements can be substituted for the ones set forth above such that they perform in substantially the same manner in substantially the same way for achieving substantially the same result.

It is believed that the system and method of the present invention and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A drainage bag support comprising:
a belt having a means for engaging with an animate object, the belt having an inwardly facing surface that rests against the animate object and an outwardly facing surface facing away from the animate object;
at least one fastening station positioned on the outwardly facing surface of the belt; and
one or more means for fastening one or more drainage bags to one or more of the fastening stations, thereby supporting the drainage bags by the belt.

2. The drainage bag support of claim 1, wherein the belt has a first end and a distal second end and the means for engaging is hook and loop material on the outwardly facing surface of the first end and mating hook and loop material on the inwardly facing surface of the second end.

3. The drainage bag support of claim 1, wherein the belt is a continuous loop of a stretchable material and the means for engaging includes tension provided by the stretchable material after the belt is stretched to engage with the animate object.

4. The drainage bag support of claim 1, wherein the animate object is a waist of a person.

5. The drainage bag support of claim 1, wherein the means for fastening is a safety pin.

6. The drainage bag support of claim 1, wherein the means for fastening is a clip.

7. The drainage bag support of claim 1, wherein the means for fastening is a loop of material.

8. The drainage bag support of claim 7, wherein an end of the loop of material passes through the fastening station and removably attaches to a mid section of the loop of material by another set of hook and loop material.

9. A method of supporting a drainage bag, the method comprising:
providing a drainage bag support, the drainage bag support having a belt, the belt made of a continuous loop of a stretchable material, the belt having an inwardly facing surface facing toward the center of the continuous loop and an outwardly facing surface facing away from the center of the continuous loop; the drainage bag support also having at least one fastening station positioned on the outwardly facing surface of the belt;
stretching the belt to engage the belt with an area of an animate object;
releasing the belt, the belt therefore contracting around the area of the animate object; and
affixing one or more drainage bag to one or more of the fastening stations.

10. The method of claim 9, wherein the animate object is a person and the step of stretching includes stretching the belt enough to clear legs and a buttock of the person.

11. The method of claim 9, wherein the step of affixing uses a clip to affix an appendage of the drainage bags to one or more of the fastening stations.

12. The method of claim 9, wherein the step of affixing uses a safety pin to affix an appendage of the drainage bags to one or more of the fastening stations.

13. The method of claim 9, wherein the step of affixing uses a loop of material to affix an appendage of the drainage bags to one or more of the fastening stations.

14. A method of supporting a drainage bag, the method comprising:
providing a drainage bag support, the drainage bag support having a belt, the belt having an inwardly facing surface and an outwardly facing surface facing opposite of the inwardly facing surface; the belt having hook and loop material on the outwardly facing surface at a first end of the belt and the belt having mating hook and loop material on the inwardly facing surface at a second end of the belt, the drainage bag support also having at least one fastening station positioned on the outwardly facing surface of the belt;
passing the belt around an area of an animate object;
engaging the hook and loop material with the mating hook and loop material, thereby securing the belt around the area of the animate object; and
affixing one or more drainage bag to one or more of the fastening stations.

15. The method of claim 14, wherein the animate object is a person and the step of engaging includes passing the belt around a waist of the person.

16. The method of claim 14, wherein the step of affixing uses a clip to affix an appendage of the drainage bags to one or more of the fastening stations.

17. The method of claim 14, wherein the step of affixing uses a safety pin to affix an appendage of the drainage bags to one or more of the fastening stations.

18. The method of claim 14, wherein the step of affixing uses a loop of material to affix an appendage of the drainage bags to one or more of the fastening stations.
